(19) United States
(12) Patent Application Publication
(10) Pub. No.: US 2022/0019892 A1
(43) Pub. Date: Jan. 20, 2022

(54) DIALYSIS EVENT PREDICTION

(71) Applicant: NEC Laboratories America, Inc., Princeton, NJ (US)

(72) Inventors: Jingchao Ni, Princeton, NJ (US); Wei Cheng, Princeton Junction, NJ (US); Haifeng Chen, West Windsor, NJ (US); Bo Zong, West Windsor, NJ (US)

(21) Appl. No.: 17/379,078

(22) Filed: Jul. 19, 2021

Related U.S. Application Data
(60) Provisional application No. 63/053,839, filed on Jul. 20, 2020.

(51) Int. Cl. G06N 3/08; G06N 7/00; G16H 20/40

(52) U.S. Cl. CPC G06N 3/08; G16H 20/40; G06N 7/005

(57) ABSTRACT

A method for training a predictive model includes training a dual-channel neural network model, which includes a static channel to process static information and a dynamic channel to process temporal information, to generate a probability score that characterizes a likelihood of a health event occurring during a dialysis procedure, based on static profile information and temporal measurement information. An augmented model is trained to generate an importance score associated with the probability score, based on the static profile information and the temporal measurement information.

### RELATED APPLICATION INFORMATION

This application claims priority to 63/053,839, filed on Jul. 20, 2020, incorporated herein by reference in its entirety.

### BACKGROUND

### Technical Field

The present invention relates to medical diagnosis and treatment, and, more particularly, to the use of machine learning in predicting and preventing adverse events during dialysis.

### Description of the Related Art

Hemodialysis is a process of purifying the blood of a patient whose kidneys are not working normally. Dialysis patients are at a high risk of cardiovascular disease, and other problems, which can involve intensive management of blood pressure, anemia, mineral metabolism, and other factors. Dialysis patients may therefore encounter health events, such as low blood pressure and leg cramps, during dialysis.

### SUMMARY

A method for training a predictive model includes training a dual-channel neural network model, which includes a static channel to process static information and a dynamic channel to process temporal information, to generate a probability score that characterizes a likelihood of a health event occurring during a dialysis procedure, based on static profile information and temporal measurement information. An augmented model is trained to generate an importance score associated with the probability score, based on the static profile information and the temporal measurement information.

A system for training a predictive model includes a hardware processor and a memory that stores a computer program product. When executed by the hardware processor, the computer program product causes the hardware processor to train a dual-channel neural network model, which includes a static channel to process static information and a dynamic channel to process temporal information, to generate a probability score that characterizes a likelihood of a health event occurring during a dialysis procedure, based on static profile information and temporal measurement information. An augmented model is trained to generate an importance score associated with the probability score, based on the static profile information and the temporal measurement information.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram of a patient undergoing dialysis, where the likelihood of a health event occurring during dialysis is predicted and used to guide treatment, in accordance with an embodiment of the present invention;

FIG. 2 is a block/flow diagram of a method for predicting a likelihood of a health event occurring during dialysis and adjusting treatment accordingly, in accordance with an embodiment of the present invention;

FIG. 3 is a block/flow diagram of a method of determining the likelihood of a health event occurring during dialysis, in accordance with an embodiment of the present invention;

FIG. 4 is a block diagram of a machine learning model that determines the likelihood of a health event occurring during dialysis, in accordance with an embodiment of the present invention;

FIG. 5 is a block diagram of a computing device configured to predict health events during dialysis, in accordance with an embodiment of the present invention; and

FIG. 6 is a diagram of a neural network model that may be used in the prediction of the likelihood of a health event occurring during dialysis, in accordance with an embodiment of the present invention.

### DETAILED DESCRIPTION OF PREFERRED EMBODIMENTS

To predict whether a health event may occur during a dialysis session, machine learning is used to generate an automatic, high-quality, interpretable prediction score. Toward this end, information collected in the days before the dialysis session can be used, shortly before the session begins, to predict whether a health event occur, using a trained machine learning model. The model may consider historical patient information, dialysis measurements, blood test measurements, and cardiothoracic ratio, for example, to provide a simple output score.

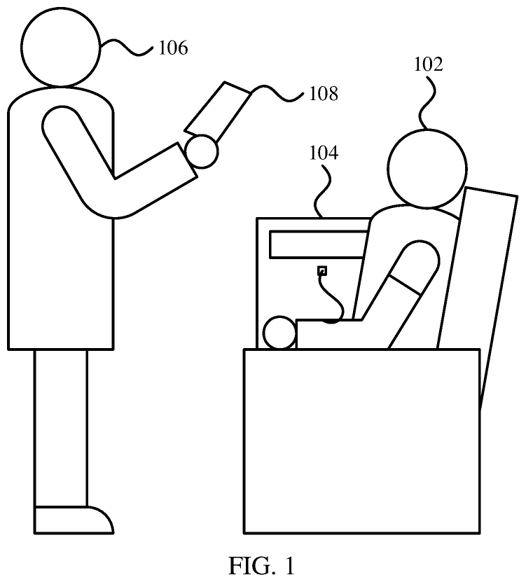

Referring now in detail to the figures in which like numerals represent the same or similar elements and initially to FIG. 1, a patient 102 is shown during a hemodialysis (also known simply as “dialysis”) session. During dialysis, a dialysis machine 104 automatically draws the patient's blood, processes and purifies the blood, and then reintroduces the purified blood to the patient's body. Dialysis can take as long as four hours to complete, and may be performed every three days, though other durations and periods are contemplated.

Before the patient undergoes dialysis, a medical professional 106 reviews a recommendation 108 that includes a prediction score. This prediction score indicates a likelihood that a health event will occur during the dialysis session. It is specifically contemplated that this recommendation may be made before the dialysis session begins, so that treatment can be adjusted.

The recommendation is based on a variety of input information. Part of that information includes a static profile of the patient, for example including information such as age, sex, starting time of dialysis, etc. The information also includes dynamic data, such as dialysis measurement records, which may be taken at every dialysis session, blood pressure, weight, venous pressure, blood test measurements, and cardiothoracic ratio (CTR). The blood test measurements may be taken regularly, for example at a frequency of twice per month, and may measure such factors as albumin, glucose, and platelet count. The CTR may also be taken regularly, for example at a frequency of once per month. The dynamic information may be modeled as time series over their respective frequencies.

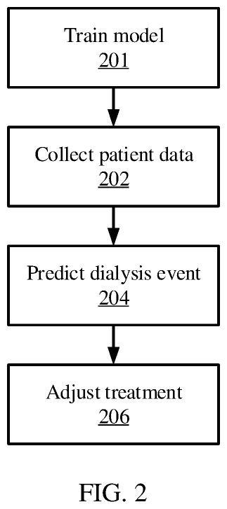

Referring now to FIG. 2, a method of predicting and preventing dialysis events is shown. Block 201 trains a dual-channel predictive model to identify a likelihood of a dialysis event, based on historical patient data. Training may be performed using an optimizer with a regression loss function:

$$l = \frac{1}{N} {\sum }_{i = 1}^{N} {‖ {\hat{y}}_{1} - {y}_{1} ‖}_{2}^{2} + λ {‖ θ ‖}_{2}^{2}$$

where yᵢ is a binary-valued true indicator of an incidence of an event for an i^{th} sample in the training data, ŷᵢ is real-valued output vector of the predictive model, θ represents model parameters, and λ is a hyperparameter that controls the regularization of model parameters, to avoid overfitting. Training may include training of the predictive model to obtain the predictive scores ŷᵢ, as well as aggregating static and temporal features to obtain concatenated features, using ŷ as soft labels to train an augmented model for finding importance scores.

Block 202 collects new patient data, which may include static information and dynamic information, as described above. Some patient data collection may be performed on an ongoing basis, whereas other data may be performed periodically, or only once. For example, patient data may be collected at each dialysis appointment, but may also be collected at home by the patient themselves, using any appropriate measurement device.

Block 204 predicts a dialysis health event, using the collected patient data. As will be described in greater detail below, prediction may use a neural network-based system that uses a dual-channel architecture, integrating the collected patient data to generate a prediction score. The prediction may include an interpretation of the prediction score, for example by attaching an importance score to different features of the patient data. The dual-channel system handles static features, low-frequency temporal features, and high-frequency temporal features for join representation learning. An attention mechanism can generate attention scores that improve prediction importance, and that highlight important time steps in the patient data for interpretation.

Once the prediction is generated, it can be used in block 206 to adjust the patient's treatment. For example, the dialysis may be delayed until health care professionals can determine the cause of an increased likelihood of a health event. In particular, the features that are indicated as being important to the determination of the score may be used to indicate particular measurements that are related to the high likelihood of an event. A threshold may be used to trigger action, with a probability score that is above the threshold being associated with a preventative action, and probability scores below the threshold indicating that it is safe to proceed with dialysis. For example, the threshold can be selected to give a maximal value for the difference between true positive rates (the frequency that health events are correctly predicted) and false positive rates (the frequency that health events are predicted, but do not occur). Thus, the threshold may be determined empirically, and may also be determined based on expert domain knowledge.

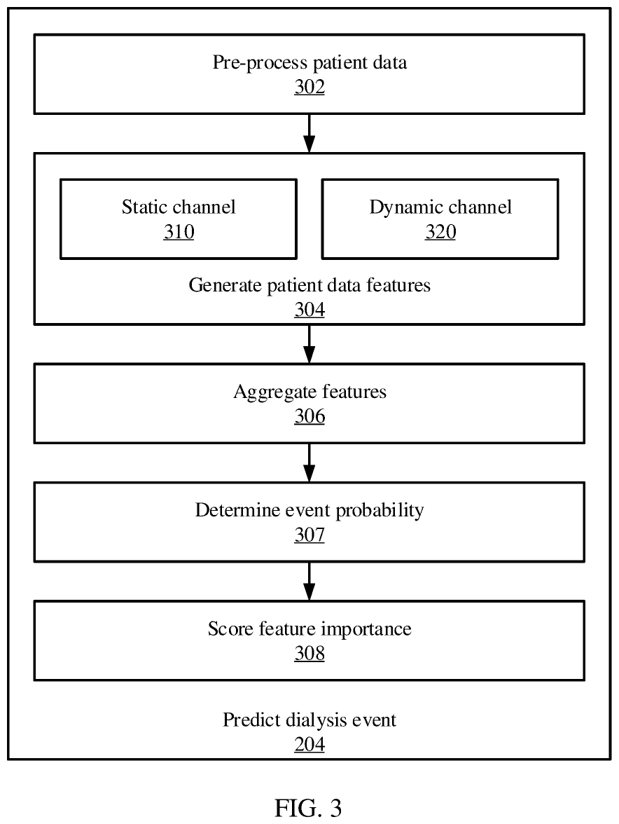

Referring now to FIG. 3, additional detail is provided regarding the prediction of dialysis events in block 204. Block 302 pre-processes the patient data. For example, patient data may include a patient history profile, dialysis measurements, blood test measurements, and past event incidences. This information may be stored in, e.g., a spreadsheet, where each row may indicate a particular data of a hospital visit by the patient, associated with one or more measurements. Each column may then indicate a particular feature, such as some indicator metric in the dialysis measurements (e.g., blood pressure, weight, venous pressure, etc.). Since different features will have different frequencies of measurement, some entries in the spreadsheet (or other data representation) may be blank, indicating that a particular feature was not measured at a particular time.

Patient data pre-processing 302 extracts parts of the data from the source data, removing noisy information and filling in missing values using, for example, an interpolated value that may be calculated according to a previous and subsequent measurement, or by using an average value of the feature across the patient's history.

The pre-processing 302 may determine a time window of width w to segment time series data. Each time window may generate a sample X from time step T−w to time step T, and associates it with an event label Y at a time step T+1. Samples may then focus on the features in the closest dates to a potential future event. Because different features may have different frequencies, different types of information may be sampled differently. For example, all dialysis measurements within a time window may be included, whereas blood test measurements may only be considered on the closest date to the time window. For example, because blood tests may be performed at a relatively low frequency (e.g., biweekly) compared to dialysis measurements (e.g., three days per week), it is possible that a window of a certain length will not include any blood test measurement. In such a case, to ensure that every window has access to blood test information, the blood test closest to the window's starting time (or ending time) may be used. The time window may slide from the beginning of data collection to the last recorded date to generate multiple samples.

After the samples are generated, pre-processing 302 may normalize the samples using, e.g., Gaussian normalization, such that the features of the training samples have a mean value of 0 and a variance of 1. Normalization facilitates stability in the following steps. To normalize new samples during operation, they may use the mean and variance obtained from the training data.

Block 304 generates features from the patient data. This prediction may include two channels: a static channel 310 for processing low-frequency temporal features. and a temporal channel 320 for processing high-frequency temporal features.

The static features (including fixed features and temporal features that have a frequency of update that is lower than a threshold) are represented by a vector xₛ. The static channel 310 may include a multilayer perceptron (MLP) to encode the information in xₛ in a compact representation hₛ=f_{MLP}(xₛ), where f_{MLP}(⋅) may be multiple layers of fully connected neural network, as described in greater detail below, with the form wₛxₛ+bₛ, where Wₛ and bₛ are model parameters to be trained. After this step, the output hₛ may be a compact representation of the static features, which may be integrated with representations from temporal channels for prediction.

The temporal channel 320 may include multiple long short-term memory (LSTM) layers for processing temporal features. The temporal features may be represented by a sequence of vectors x₁, . . . , x_{T}, and the LSTM layers may output a sequence of compact representations h₁, . . . , h_{T}=f_{LSTM}(x₁, . . . , x_{T}), where f_{LSTM}(⋅) may have multiple layers of LSTM units, including trainable model parameters. The LSTM units may be extended to bi-directional LSTM units, to encode information from both temporal directions.

The representations h₁, . . . , h_{T} may be sent to an attention layer for combination. The attention layer may calculate a temporal importance score, such as an attention weight αₜ for each time step by:

eₜ=w_{α} tan h(W_{α}hₜ) for t=1, . . . ,T

αₜ=softmax(eₜ) for t=1, . . . ,T

where W_{α} and w_{α} are model parameters to learn. After this, Σₜ₌₁^{T}, αₜ=1.

The compact temporal representations may then be combined through attention weights by:

$${h}_{d} = {\sum }_{t = 1}^{T} {α}_{t} {h}_{t}$$

The compact representation h_{d} includes all temporal features x₁, . . . , x_{T} and is the output of the temporal channel 320.

After the static representation hₛ and the temporal representation h_{d} have been determined in block 304, block 306 aggregate the features. One type of aggregation may be a concatenation of the temporal features x₁, . . . , x_{T} to obtain a long vector, which may be concatenated with the static features xₛ to form a feature vector \hat{x}. Another option for aggregation is to generate hidden features and statistics from the temporal features, for example using statistics (e.g., mean, variance, max, and min of the temporal features over time), temporal change measurements (e.g., the differences between temporal features measured at different time steps), and generating temporal feature to represent days of dialysis duration (e.g., the difference between a present date and an initial dialysis date). These generated features may be aggregated together with the concatenated temporal features and static features, to be included in \hat{x}.

Block 307 determines the probability of events, for example using an MLP, by:

ŷ=f_{MLP}([hₛ,h_{d}])

where ŷ is a score that indicates the probability of the incidence of an event.

It can be difficult to obtain importance scores, due to the deep recurrent structure of the probability determination. Block 308 therefore augments the predictive model with a simple interpretable model, using knowledge distillation to achieve both high accuracy and interpretability. Gradient boosting trees may be used as the augmented model, which may output feature importance scores. Then the knowledge from the predictive model may be used to train the gradient boosting trees. The training of the augmented model may use aggregated features xₛ and x₁, . . . , x_{T} to obtain a concatenated feature \hat{x}, using ŷ as soft labels to train a gradient boosting tree regressor.

The feature importance scores may represent how each feature helps shape the class boundary between normal segments and events. A high importance score may indicate that a feature is more likely to separate normal segments and events well. However, individual features may not be enough to separate two classes. As a result, a set of features that are highly ranked by importance score may be interpreted as the features that are most helpful to differentiate normal segments and events. These identified features may be interpreted by a domain expert to provide an explanation for how the model makes predictions.

During the training of block 201, a loss function for training the gradient boosting tree regressor may be expressed as:

$$l = \frac{1}{N} {\sum }_{i = 1}^{N} {‖ {\hat{y}}_{i} - {( {\hat{y}}_{new} )}_{i} ‖}_{2}^{2}$$

where (ŷ_{new})ᵢ is the newly obtained prediction score for the i^{th} sample. After training, the augmented model can output feature importance scores. Notably, either ŷ or ŷ_{new} may be used as prediction scores.

During testing, given a new sample, either ŷ from the probability computation of block 307 or ŷ_{new} from the importance scoring of block 308 may be used as prediction scores. The gradient boosting trees may be used to achieve feature importance scores. The feature importance may be interpreted as a post hoc explanation of the probability score, and provides an approximation to interpret the important features in the predictions of the probability determination.

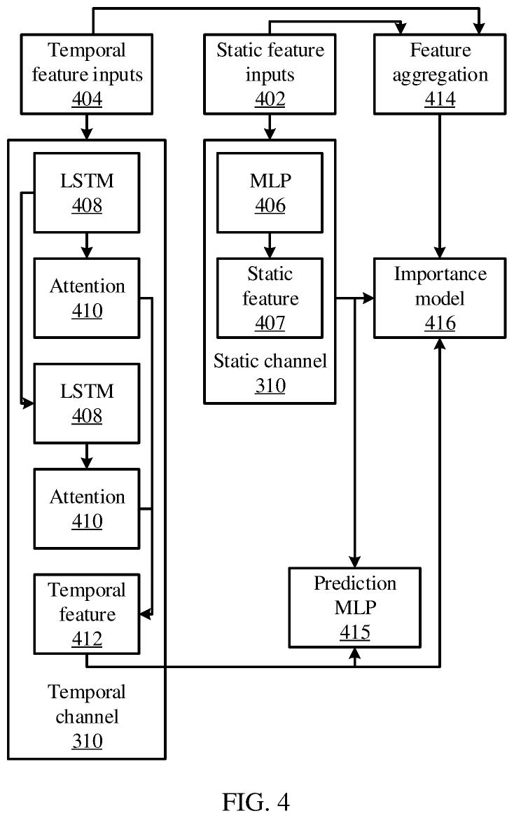

Referring now to FIG. 4, the structure of an exemplary prediction and importance model is shown. Static feature inputs 402 and temporal feature inputs 404 are provided to the model. The static feature inputs 402 are processed by static channel 310, which uses MLP 406 to generate static features 407. The temporal feature inputs 404 are processed by temporal channel 320, which uses a series of LSTM layers 408, each of which feeds into the next and each of which generates an output through attention 410. It should be noted that any appropriate of LSTM layers 408 and respective attentions 410 may be used, and that the LSTM layers 408 may be implemented as bi-directional LSTM layers. The attention outputs are collected into a temporal feature 412.

The input features 402 and 404 are meanwhile aggregated at feature aggregation 414. The aggregated input features, as well as the static output features 407 and the temporal output features 412 may be used as inputs to importance model 416, which identifies an importance score. The static output features 407 and the temporal output features 412 may also be processed by a prediction model 415, for example implemented as an MLP, to generate a prediction probability.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

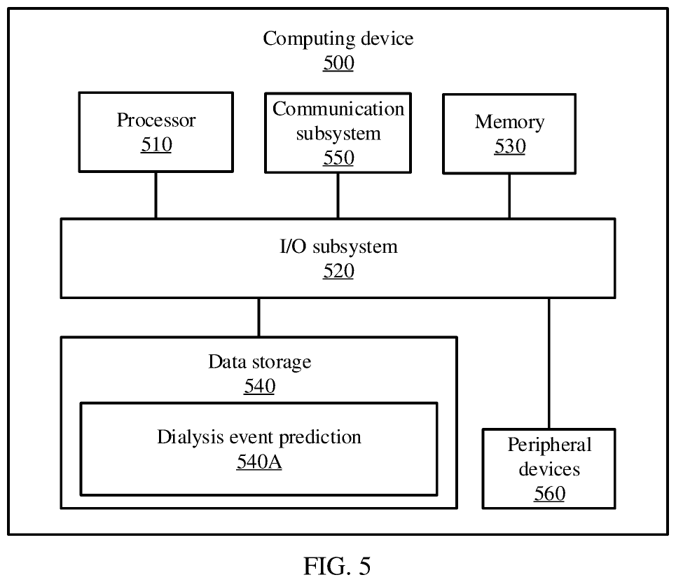

FIG. 5 is a block diagram showing an exemplary computing device 500, in accordance with an embodiment of the present invention. The computing device 500 is configured to perform dialysis event prediction.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally, or alternatively, the computing device 500 may be embodied as a one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for dialysis event prediction. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

As employed herein, the term “hardware processor subsystem” or “hardware processor” can refer to a processor, memory (including RAM, cache(s), and so forth), software (including memory management software) or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), FPGAs, and/or PLAs.

These and other variations of a hardware processor subsystem are also contemplated in accordance with embodiments of the present invention.

An artificial neural network (ANN) is an information processing system that is inspired by biological nervous systems, such as the brain. The key element of ANNs is the structure of the information processing system, which includes a large number of highly interconnected processing elements (called “neurons”) working in parallel to solve specific problems. ANNs are furthermore trained using a set of training data, with learning that involves adjustments to weights that exist between the neurons. An ANN is configured for a specific application, such as pattern recognition or data classification, through such a learning process.

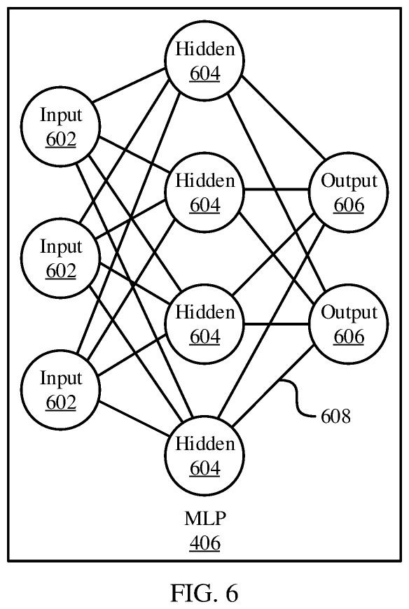

Referring now to FIG. 6, a generalized diagram of a neural network is shown, as an MLP such as may be used in static channel 310. Although a specific structure of an ANN is shown, having three layers and a set number of fully connected neurons, it should be understood that this is intended solely for the purpose of illustration. In practice, the present embodiments may take any appropriate form, including any number of layers and any pattern or patterns of connections therebetween.

ANNs demonstrate an ability to derive meaning from complicated or imprecise data and can be used to extract patterns and detect trends that are too complex to be detected by humans or other computer-based systems. The structure of a neural network is known generally to have input neurons 602 that provide information to one or more “hidden” neurons 604. Connections 608 between the input neurons 602 and hidden neurons 604 are weighted, and these weighted inputs are then processed by the hidden neurons 604 according to some function in the hidden neurons 604. There can be any number of layers of hidden neurons 604, and as well as neurons that perform different functions. There exist different neural network structures as well, such as a convolutional neural network, a maxout network, etc., which may vary according to the structure and function of the hidden layers, as well as the pattern of weights between the layers. The individual layers may perform particular functions, and may include convolutional layers, pooling layers, fully connected layers, softmax layers, or any other appropriate type of neural network layer. Finally, a set of output neurons 606 accepts and processes weighted input from the last set of hidden neurons 604.

This represents a “feed-forward” computation, where information propagates from input neurons 602 to the output neurons 606. Upon completion of a feed-forward computation, the output is compared to a desired output available from training data. The error relative to the training data is then processed in “backpropagation” computation, where the hidden neurons 604 and input neurons 602 receive information regarding the error propagating backward from the output neurons 606. Once the backward error propagation has been completed, weight updates are performed, with the weighted connections 608 being updated to account for the received error. It should be noted that the three modes of operation, feed forward, back propagation, and weight update, do not overlap with one another. This represents just one variety of ANN computation, and that any appropriate form of computation may be used instead.

To train an ANN, training data can be divided into a training set and a testing set. The training data includes pairs of an input and a known output. During training, the inputs of the training set are fed into the ANN using feed-forward propagation. After each input, the output of the ANN is compared to the respective known output. Discrepancies between the output of the ANN and the known output that is associated with that particular input are used to generate an error value, which may be backpropagated through the ANN, after which the weight values of the ANN may be updated. This process continues until the pairs in the training set are exhausted.

After the training has been completed, the ANN may be tested against the testing set, to ensure that the training has not resulted in overfitting. If the ANN can generalize to new inputs, beyond those which it was already trained on, then it is ready for use. If the ANN does not accurately reproduce the known outputs of the testing set, then additional training data may be needed, or hyperparameters of the ANN may need to be adjusted.

ANNs may be implemented in software, hardware, or a combination of the two. For example, each weight 608 may be characterized as a weight value that is stored in a computer memory, and the activation function of each neuron may be implemented by a computer processor. The weight value may store any appropriate data value, such as a real number, a binary value, or a value selected from a fixed number of possibilities, that is multiplied against the relevant neuron outputs.

Different types of ANN are described herein. An MLP may use fully connected layers, where each neuron on one layer is connected to every layer in a subsequent layer. Other types of networks, such as recurrent neural networks (RNNs) and LSTM networks are also contemplated. Recurrent neural networks (RNNs) may be used to process sequences of information, such as an ordered series of feature vectors. This makes RNNs well suited to text processing and time series processing, where information is naturally sequential. Each neuron in an RNN receives two inputs: a new input from a previous layer, and a previous input from the current layer. An RNN layer thereby maintains information about the state of the sequence from one input to the next.

LSTM networks are a variety of RNN that store information within the LSTM neurons for future use. Use of the memory may be controlled by the neuron's activation function. The use of this memory helps preserve gradient information during backpropagation.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method for training a predictive model, comprising:
training, using a hardware processor, a dual-channel neural network model, which includes a static channel to process static information and a dynamic channel to process temporal information, to generate a probability score that characterizes a likelihood of a health event occurring during a dialysis procedure, based on static profile information and temporal measurement information; and
training an augmented model to generate an importance score associated with the probability score, based on the static profile information and the temporal measurement information.

2. The method of claim 1, wherein the dynamic channel includes a series of long short-term memory layers.

3. The method of claim 1, wherein the static channel includes a multi-layer perceptron.

4. The method of claim 1, wherein training the dual-channel neural network model includes training a prediction multi-layer perceptron to determine the probability score, based on a static feature from the static channel and a temporal feature from the dynamic channel.

5. The method of claim 1, wherein training the augmented model includes training gradient boosting trees to output feature importance scores for a static feature from the static channel and a temporal feature from the dynamic channel.

6. The method of claim 5, wherein training the augmented model includes minimizing a loss function for a gradient boosting tree regressor:
$$l = \frac{1}{N} {\sum }_{i = 1}^{N} {‖ {\hat{y}}_{i} - {( {\hat{y}}_{new} )}_{i} ‖}_{2}^{2}$$
where N is a number of samples, ŷᵢ is the probability score for the i^{th}, (ŷ_{new})ᵢ is a newly obtained probability score for an i^{th} sample.

7. The method of claim 1, wherein the temporal information includes time series measurements made of one or more characteristics of a patient.

8. The method of claim 7, further comprising pre-processing the temporal information, including splitting time series measurements into windows of a predetermined length.

9. The method of claim 8, wherein pre-processing the temporal information includes adding, as part of a first window, a measurement for a first characteristic that was made outside of the first window, responsive to a determination that the window includes no measurements for the first characteristic.

10. The method of claim 7, wherein the temporal information includes blood test information, dialysis measurements, and past event incidences.

11. A system for training a predictive model, comprising:
a hardware processor; and
a memory that stores a computer program product, which, when executed by the hardware processor, causes the hardware processor to:
train a dual-channel neural network model, which includes a static channel to process static information and a dynamic channel to process temporal information, to generate a probability score that characterizes a likelihood of a health event occurring during a dialysis procedure, based on static profile information and temporal measurement information; and
train an augmented model to generate an importance score associated with the probability score, based on the static profile information and the temporal measurement information.

12. The system of claim 11, wherein the dynamic channel includes a series of long short-term memory layers.

13. The system of claim 11, wherein the static channel includes a multi-layer perceptron.

14. The system of claim 11, wherein the computer program product further causes the hardware processor to train a prediction multi-layer perceptron to determine the probability score, based on a static feature from the static channel and a temporal feature from the dynamic channel.

15. The system of claim 11, wherein the computer program product further causes the hardware processor to train gradient boosting trees to output feature importance scores for a static feature from the static channel and a temporal feature from the dynamic channel.

16. The system of claim 15, wherein the computer program product further causes the hardware processor to minimize a loss function for a gradient boosting tree regressor:
$$l = \frac{1}{N} {\sum }_{i = 1}^{N} {‖ {\hat{y}}_{i} - {( {\hat{y}}_{new} )}_{i} ‖}_{2}^{2}$$
where N is a number of samples, ŷᵢ is the probability score for the i^{th}, (y_{new})ᵢ is a newly obtained probability score for an i^{th} sample.

17. The system of claim 11, wherein the temporal information includes time series measurements made of one or more characteristics of a patient.

18. The system of claim 17, wherein the computer program product further causes the hardware processor to pre-process the temporal information, including splitting time series measurements into windows of a predetermined length.

19. The system of claim 18, wherein the computer program product further causes the hardware processor to add, as part of a first window, a measurement for a first characteristic that was made outside of the first window, responsive to a determination that the window includes no measurements for the first characteristic.

20. The system of claim 17, wherein the temporal information includes blood test information, dialysis measurements, and past event incidences.
